(19) United States
(12) Patent Application Publication
(10) Pub. No.: US 2023/0174450 A1
(43) Pub. Date: Jun. 8, 2023

(54) PROCESS AND SYSTEM FOR PURIFICATION OF CITRIC ACID

(71) Applicant: ENTEGRIS, INC., Billerica, MA (US)

(72) Inventors: Yuxin Song, Shanghai (CN); Xianxin Wu, Shanghai (CN); Chao Zhou, Shanghai (CN); Yu Dong, Shanghai (CN); Ping Jiang, Beijing (CN); ZhengZheng Du, Shanghai (CN); Zicheng Song, Shanghai (CN)

(21) Appl. No.: 17/924,264

(22) PCT Filed: Jun. 8, 2020
(86) PCT No.: PCT/CN2020/094910

§ 371 Date: Nov. 9, 2022

(51) Int. Cl. C07C 51/42; B01D 61/00; B01D 37/04

(52) U.S. Cl. CPC C07C 51/42; B01D 37/04; B01D 61/00; B01D 2313/44

(57) ABSTRACT

Citric acid is purified to remove metal ions through a two-step filtration process. A first filter is used to perform a first filtration, then a second filter is used to perform a second filtration on citric acid solution that has been subject to the first filtration. The first and second filters can include the same filter membrane material. The filter used as the first filter can be a relatively dirtier, more loaded filter compared to the filter used as the second filter. The first filtration can be performed over four hours of recirculating the citric acid solution through the first filter, and the second filtration can be performed over approximately two and one half hours of recirculating the citric acid solution through the second filter. Such a purification process can remove calcium and magnesium ions to render citric acid suitable as a cleaning solution in semiconductor processing.

### FIELD

This disclosure is directed to purification of citric acid, particularly removal of metals using a plurality of filters.

### BACKGROUND

Citric acid can be used for cleaning during semiconductor processing. To be suitable for such use, the citric acid must be sufficiently pure, with low levels of contaminants such as metals including calcium and magnesium.

Filters can be used to remove the contaminants. However, filter membranes suitable for removing metallic contaminants can quickly become loaded to a point at which effectiveness falls below acceptable thresholds. This can lead to frequent replacement of filters for preparing citric acid, resulting in high costs for processing of citric acid, waste of components, and slow processing to accommodate the required filter changes.

### SUMMARY

This disclosure is directed to purification of citric acid, particularly removal of metals using a plurality of filters.

The purification of citric acid by embodiments described herein allows sufficiently pure citric acid to be produced even as the loading of the filtration membranes increases, improving the efficiency and reducing the number of filters required to purify a set quantity of citric acid. The purification can be done in an order allowing filtration membranes to be replaced while citric acid is being processed by another membrane, allowing the process to continue without interruption.

In an embodiment, a method for purifying citric acid includes performing a first filtration of citric acid using a first filter and performing a second filtration. The second filtration is performed on the citric acid following the first filtration, using a second filter separate from the first filter.

In an embodiment, the first filter and the second filter each include a same filter membrane material.

In an embodiment, the method further includes selecting a filter having comparatively greater trapped metal ions to be used as the first filter, and a filter having comparatively fewer trapped metal ions to be used as the second filter. In an embodiment, the method further includes replacing the first filter after completion of the first filtration.

In an embodiment, performing the first filtration includes recirculating the citric acid through a fluid circuit including the first filter for approximately 4 hours. In an embodiment, performing the second filtration includes recirculating the citric acid through a fluid circuit including the second filter for approximately 2.5 hours.

In an embodiment, the citric acid is a 30% solution of citric acid. In an embodiment, after the second filtration, the citric acid includes less than 5 parts per billion (ppb) of magnesium ions, and less than 10 ppb of calcium ions.

In an embodiment, a citric acid purification system includes a tank configured to hold a citric acid solution, a first filter configured to remove metal ions from the citric acid solution, a second filter configured to remove metal ions from the citric acid solution, in parallel with the first filter, one or more valves configured to direct a flow of a fluid from the tank to one of the first filter or the second filter, a return line, configured to convey the fluid from the one of the first filter or the second filter to the tank, and a controller. The controller is configured to control the one or more valves such that the citric acid solution is first circulated through one of the first filter or the second filter for a first predetermined amount of time, and then the citric acid solution is circulated through the other of the first filter or the second filter for a second predetermined amount of time.

In an embodiment, the citric acid purification system further includes an outlet line including an outlet valve, connected to the return line.

In an embodiment, the first filter and the second filter each include a same membrane material. In an embodiment, the first filter is included in a first replaceable filter cartridge and the second filter is included in a second replaceable filter cartridge.

In an embodiment, the first predetermined amount of time is approximately 4 hours and the second predetermined amount of time is approximately 2.5 hours.

In an embodiment, the controller is further configured to determine when to replace one or more of the first filter and the second filter based on a loading of the one or more of the first filter and the second filter. In an embodiment, the loading of the one or more of the first filter and the second filter is determined based on a number of purification cycles performed since a most recent replacement of the one or more of the first filter and the second filter.

### DRAWINGS

FIG. 1 shows a flowchart of a method for purifying citric acid.

FIG. 2 shows a schematic view of a purification system according to an embodiment.

### DETAILED DESCRIPTION

This disclosure is directed to purification of citric acid, particularly removal of metals using a plurality of filters.

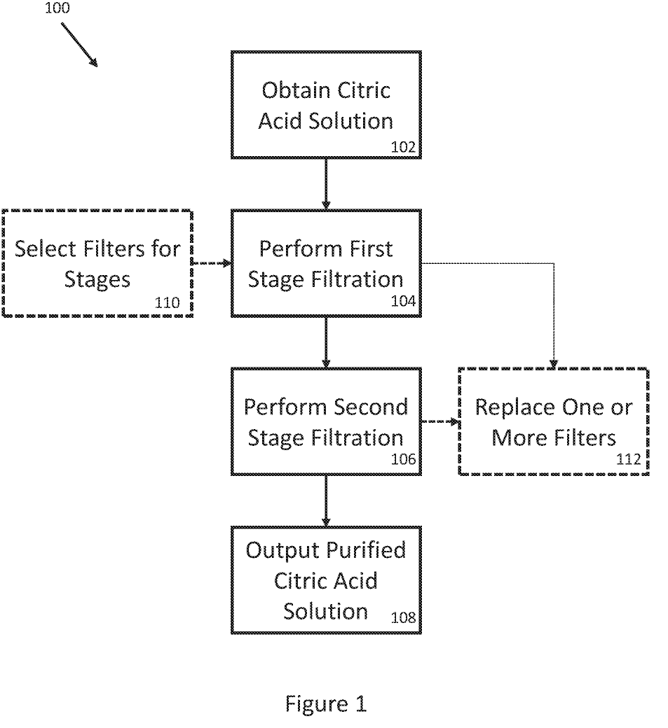

FIG. 1 shows a flowchart of a method for purifying citric acid. In method 100, a citric acid source is obtained at 102. The citric acid source is processed with a first stage filtration 104. After the first stage filtration at 104, the citric acid source is processed with a second stage filtration 106. After the second stage filtration at 106, the purified citric acid is output at 108.

A citric acid solution is obtained at 102. The citric acid source can be, for example, a 30% food grade solution of citric acid. The citric acid source can contain metal ions such as, for example, magnesium, calcium, iron, and zinc ions. The citric acid source can be added to a tank of a processing system when it is obtained at 102. The citric acid solution can be, for example, a citric acid solution having a concentration of less than 35% citric acid.

The citric acid solution is processed with a first stage filtration at 104. The first stage filtration is performed using a filter including a membrane configured to remove metal ions from the citric acid solution. The first filter can be included in a replaceable filter cartridge. In an embodiment, the first filter can be a commercially available filter such as the Protego® Plus LT filter available from Entegris, Inc. The first stage filtration at 104 can include recirculation of the citric acid solution through the first filter for a first predetermined amount of time. The first predetermined amount of time can be based on the selected filter being used as the first filter and the properties of the citric acid solution obtained at 102. In an embodiment, the first predetermined amount of time can be based on the metal ion concentration of the solution. In an embodiment, the first predetermined amount of time can be based on a volume of citric acid solution to be processed. In an embodiment, the first predetermined amount of time is between approximately one half-hour and approximately four hours. In an embodiment, the first predetermined amount of time is approximately four hours.

After the first stage filtration at 104, the citric acid solution is processed with a second stage filtration at 106. The second stage filtration is performed at 106 using a second filter, separate from the first filter used in the first stage filtration at 104. The second filter can also be a filter including a membrane configured to remove metal ions from the citric acid solution. In an embodiment, the second filter uses the same membrane material as the first filter. In an embodiment, the second filter is included in replaceable filter cartridge. In an embodiment, the second filter is the same commercially available filter such as the Protego® Plus LT filter available from Entegris, Inc. The second stage filtration can include recirculation of the citric acid solution through the second filter for a second predetermined amount of time. The second predetermined amount of time can be based on, for example, the filter selected for use as the second filter and the concentrations of metal ions in the citric acid solution following the first filtration 104. The second predetermined amount of time can be a different amount of time from the first predetermined amount of time for the first stage filtration 104. The second predetermined amount of time can be a shorter amount of time compared to the first predetermined amount of time. In an embodiment, the second predetermined amount of time is between approximately one half-hour and approximately four hours. In an embodiment, the second predetermined amount of time is approximately two and one half hours.

After the second stage filtration at 106, the purified citric acid is output at 108. The purified citric acid can have levels of metal ions sufficiently low that the citric acid solution is suitable for use in semiconductor processing, for example as a cleaning solution for use during such processes or for tools used in such processes. In an embodiment, the purified citric acid has fewer than 10 parts per billion (ppb) each of calcium ions and iron ions, fewer than 5 ppb each of magnesium ions and aluminum ions, and fewer than 1 ppb of zinc ions. In an embodiment, the purified citric acid can be further filtered, for example using one or more particle removal filters, to remove particulate matter or other contaminants beyond the metal ions removed by method 100.

Optionally, when the method is carried out repeatedly, the particular filter for use in the first stage filtration at 104 and/or the second stage filtration at 106 can be selected at 110. In an embodiment, the filter selected to be used in the first stage filtration is a relatively dirty or loaded filter compared to the filter selected for to be used in the second stage filtration at 106. The relative loading of the filters can be determined, for example, based on a number of iterations of method 100 in which each of the filters has been used. In an embodiment, the filter having the largest number of uses since its installation is selected for use as the first filter for the first stage filtration at 104. A filter having a relatively smaller number of uses since installation can be selected for use as the second filter for the second stage filtration 106.

Optionally, following the method, one or more of the filters used therein can be replaced at 112. Replacement of the filters at 112 can be based on the effectiveness or the loading of the filters. In an embodiment, replacement of one or more of the filters can be determined based on levels of metal ions in a sample taken from the citric acid solution, for example after the first filtration at 104 or the second filtration at 106. In an embodiment, the replacement of one or more of the filters can be determined based on loading as measured by the number of filtration steps the filter has been used for in previous iterations of method 100. In an embodiment, a filter can be replaced when it has been used as a first filter for the first filtration at 104 three times. When one filter is replaced, the new filter in its place becomes the relatively cleaner, less loaded filter compared to filters that have been used for previous iterations of the method 100 with respect to the selection of filters for first and second stage filtration at 110. In an embodiment, the time at which to replace a filter can be based at least in part on a volume of solution processed using the filter. In an embodiment, the time at which to replace a replace a filter can be based at least in part on the ion concentrations in the initial solution being purified using method 100.

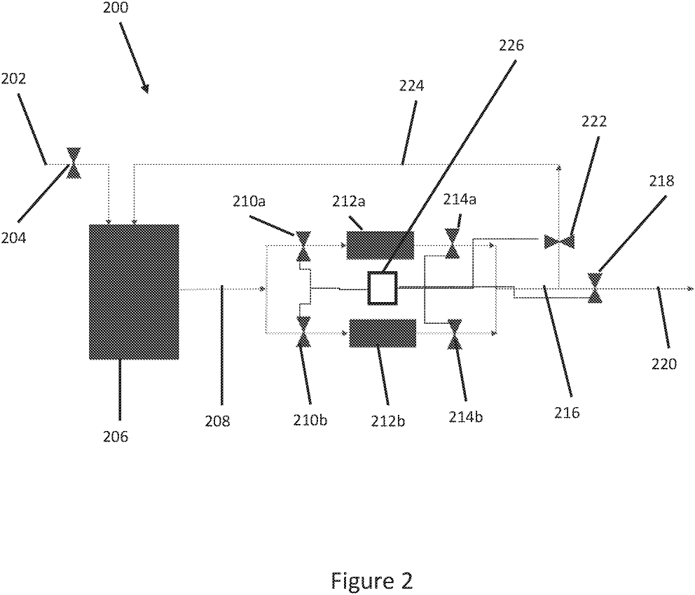

FIG. 2 shows a schematic view of a purification system according to an embodiment. Purification system 200 includes inlet 202 feeding into tank 206, with the flow controlled by inlet valve 204. Filter line 208 connects tank 206 to first filter 212a and second filter 212b, with flow to each filter 212a and 212b controlled by respective filter inlet valves 210a and 210b. Filter outlet valves 214a and 214b control the flow from, respectively, first filter 212a and second filter 212b into filter output line 216. Filter output line 216 can provide an outlet flow through system outlet valve 218 into output 220. Filter output line 216 can provide a recirculation flow through recirculation valve 222 into recirculation line 224, which is connected to tank 206. Controller 226 can control operation of the purification system 200.

Purification system 200 can be a system configured to perform both the first and second filtration steps 104 and 106 shown in FIG. 1 and described above. Purification system 200 can be used to purify a citric acid solution to remove metal ions from the citric acid solution.

Inlet 202 allows citric acid solution to be provided to tank 206. Inlet 202 can include an interface configured to receive citric acid solution from a source such as another vessel containing the citric acid solution. Inlet valve 204 can be included along a fluid line connected to inlet 202 to control flow into tank 206, for example to prohibit flow into tank 206 during an ongoing purification process, and allow tank 206 to be refilled after purified citric acid solution has been removed from tank 206.

Tank 206 is vessel configured to hold the citric acid solution. Tank 206 can include any material suitable for storing citric acid. The material of tank 206 contacting the citric acid can be resistant to corrosion or other reaction with the citric acid, and does not release contaminants or particulate matter to the citric acid solution. Non-limiting examples of materials suitable for storage of citric acid include high-density polyethylene (HDPE), poly(tetrafluoroethylene) (PTFE), and perfluoroalkoyalkane polymers (PFA). In an embodiment, tank 206 includes a drain allowing the citric acid solution to be removed from the purification system 200. In an embodiment, tank 206 includes a drain line and a valve configured to allow the draining of water from tank 206 following a cleaning process.

Filter line 208 connects tank 206 to filter inlet valves 210a and 210b. Filter line 208 includes a joint where the filter line 208 divides into branches, with one branch connecting to filter inlet valve 210a and another branch connecting to filter inlet valve 210b.

First and second filter inlet valves 210a and 210b, respectively, are valves configured to control flow from filter line 208 to first filter 212a and second filter 212b. Each of filter inlet valves 210a and 210b can be any suitable valve having a closed position prohibiting flow therethrough and an open position allowing flow therethrough. During a filtration such as first or second filtration 104 or 106 described above, one of first filter 212a and second filter 212b is in the open position and the other is in the closed position such that one of the first and second filters 212a and 212b are receiving citric acid solution from filter line 208 based on whether the purification system 200 is performing the first or second filtration step. In an embodiment, both the first and second filter inlet valves 210a and 210b can be closed when the tank 206 is being filled before a purification process performed using purification system 200.

First filter 212a is a filter that separating metal ions from a citric acid solution that is passed through. First filter 212a can be any filter suitable for use with citric acid without being damaged or resulting in contamination, and capable of trapping metal ions, including at least calcium and magnesium ions. First filter 212a can be included in a replaceable cartridge that can be swapped out, for example when first filter inlet valve 210a and first filter outlet valve 214a are closed so that no flow is reaching first filter 212a. First filter 212a can be, for example, a Protego® Plus LT filter, available from Entegris, Inc.

Second filter 212b is another filter, separate from and in parallel with first filter 212a. Second filter 212b can be any filter suitable for use with citric acid without being damaged or resulting in contamination, and capable of trapping metal ions, including at least calcium and magnesium ions. Second filter 212b can be included in a replaceable cartridge that can be swapped out, for example when second filter inlet valve 210b and second filter outlet valve 214b are closed so that no flow is reaching second filter 212b. Second filter 212b can be, for example, a Protego® Plus LT filter, available from Entegris, Inc. In an embodiment, second filter 212b includes the same filter material as first filter 212a. In an embodiment, second filter 212b is the same type of replaceable filter cartridges as first filter 212a.

First and second filter outlet valves 214a and 214b, respectively, allow flow from the first and second filters 212a and 212b into filter output line 216. The filter outlet valves 214a and 214b can each be any suitable valve having a closed position prohibiting flow therethrough and an open position allowing flow therethrough. First and second filter outlet valves 214a and 214b can respectively be closed to prevent flow from entering first and second filters 212a and 212b when either or both of those are not in use. For example, during a first filtration step such as 104, the filter outlet valve 214a and 214b corresponding to the filter being used is in the open position and the filter outlet valve 214a and 214b corresponding to the filter that is not in use is in the closed position. In another example, when the tank 206 is being emptied, the one of the filter outlet valves 214a and 214b associated with the filter used for the second filtration process can be in the open position.

Filter output line 216 is a fluid line configured to receive fluid from the filter outlet valves 214a and 214b and conveying the fluid to recirculation valve 222. Optionally, outlet valve 218 can be included along filter output line 216, connecting filter output line 216 to outlet 220. Outlet valve 218 is a valve having a closed position prohibiting flow therethrough and an open position allowing flow therethrough. Outlet 220 can allow citric acid solution to be removed from purification system 200, for example to remove samples of the citric acid for testing or to remove purified citric acid when a purification process performed by purification system 200 is complete.

Recirculation valve 222 can be any suitable valve having a closed position prohibiting flow therethrough and an open position allowing fluid to pass therethrough. Recirculation valve 222 can be in the open position during filtration steps of a purification process performed by the purification system 200. Recirculation valve 222 can be closed, for example, when purified citric acid solution is leaving the purification system 200 through outlet valve 218 and outlet 220.

Recirculation line 224 is a fluid line configured to convey fluid from the recirculation valve 222 back to tank 206. The return of fluid through recirculation line 224 allows fluid to be recirculated through one of first and second filters 212a and 212b over time, for example to carry out the first filtration at 104 and the second filtration at 106 as described above. In an embodiment, return of fluid to tank 206 by the recirculation line 224 can return fluid at the end of purification process performed by purification system 200 such that the purified citric acid solution can be drained from the tank 206.

Controller 226 can be provided to control operation of the purification system 200, for example by controlling the operation of the filter inlet valves 210a and 210b, filter outlet valves 214a and 214b, outlet valve 218, and recirculation valve 222. Controller 226 can be operatively connected to those valves such that control signals can be provided to those valves by the controller 226. The controller 226 can be configured to direct the purification system 200 to carry out the method 100 described above and shown in FIG. 1. For example, controller 226 can be configured to open and close the valves such that one of first filter 212a and second filter 212b is receiving citric acid solution during the appropriate filtration step, first filtration step at 104 or second filtration step at 106. In an embodiment, controller 226 can select one of first filter 212a or second filter 212b to be used in the first filtration 104 based on which of the first filter 212a or second filter 212b based on which of those filters is relatively dirtier or more loaded. In an embodiment, the determination of filter dirtiness/cleanliness or loading can be based on a number of purification processes in which that filter has been used. In an embodiment, the controller 226 can track a number of purification processes or batches processed since first filter 212a or second filter 212b has been most recently replaced. In an embodiment, this tracked number of processes can be used to select which of first filter 212a and second filter 212b is used in the first filtration 104 with the other being used in the second filtration 106 as described above. In an embodiment, controller 226 can further determine when to replace at least one of first filter 212a or second filter 212b based on loading or removal effectiveness of the filter. In an embodiment, removal effectiveness can be used to determine when to replace a filter based on measurements of one or more metal ions in a sample of the citric acid solution following a filtration step including that filter. In an embodiment, the measurements of the one or more metal ions are compared to threshold values for filter performance. The threshold values can be based on acceptable levels for the metal ions in order for the citric acid solution to be suitable for use as a cleaner for semiconductor processing tools or during semiconductor processing. In an embodiment, loading of the filter can be used to determine when to replace a filter based on a number of purification processes or batches of citric acid purified by that filter since its installation. In an embodiment, the number of purification processes is compared to a threshold value and the filter replaced if it has been used for a number of purification processes that matches or exceeds the threshold value. The threshold value can be determined based on measurements of removal effectiveness or filter loading over repeated purification processes. In an embodiment, the threshold includes use of the filter in three purification processes. The threshold can be in terms of amount of solution processed over time and/or initial metal ion concentrations of the solutions that are processed using the filters 212a and 212b.

Purification according to embodiments can significantly improve the effectiveness of filtration and efficient use of filters. In typical single-stage filtration of citric acid using a single filter, after processing one batch of citric acid, the loading of the filter prevents subsequent batches processed using the filter from meeting specifications for purity with respect to metal ion content. In contrast, two-stage filtration according to embodiments allows at least three batches to be processed before replacement of filters is required. The filters used in two-stage filtration achieve much higher loading, increasing the amount of metal ions each can absorb from citric acid solutions and thus increasing the amount of solution that can be processed before replacement is required. This can significantly reduce filter consumption per quantity of citric acid that is processed. Further, the advantages can be increased by using the relatively dirtier filter for the first stage filtration, and replacing only that relatively dirtier filter when replacing a filter. The resulting citric acid is electronics-grade citric acid meeting the requirements and specifications for such citric acid, including having fewer than 10 ppb of many metal ions such as calcium and iron ions, with some metal ions present only in quantities of less than 5 ppb such as magnesium and aluminum ions.

### Aspects

It is understood that any of aspects 1-8 can be combined with any of aspects 9-15.

Aspect 1. A method for purifying citric acid, comprising:
performing a first filtration of citric acid using a first filter; and
performing a second filtration, the second filtration being performed on the citric acid following the first filtration, using a second filter separate from the first filter.

Aspect 2. The method according to aspect 1, wherein the first filter and the second filter each include a same filter membrane material.

Aspect 3. The method according to aspect 2, further comprising selecting a filter having comparatively greater trapped metal ions to be used as the first filter, and a filter having comparatively fewer trapped metal ions to be used as the second filter.

Aspect 4. The method according to aspect 3, further comprising replacing the first filter after completion of the first filtration.

Aspect 5. The method according to any of aspects 1-4, wherein the performing of the first filtration includes recirculating the citric acid through a fluid circuit including the first filter for approximately 4 hours.

Aspect 6. The method according to any of aspects 1-5, wherein the performing of the second filtration includes recirculating the citric acid through a fluid circuit including the second filter for approximately 2.5 hours.

Aspect 7. The method according to any of aspects 1-6, wherein the citric acid is a 30% solution of citric acid.

Aspect 8. The method according to any of aspects 1-7, wherein after the second filtration, the citric acid includes less than 5 parts per billion (ppb) of magnesium ions, and less than 10 ppb of calcium ions.

Aspect 9. A citric acid purification system, comprising:
a tank configured to hold a citric acid solution;
a first filter configured to remove metal ions from the citric acid solution;
a second filter configured to remove metal ions from the citric acid solution, in parallel with the first filter;
one or more valves configured to direct a flow of a fluid from the tank to one of the first filter or the second filter;
a return line, configured to convey the fluid from the one of the first filter or the second filter to the tank; and
a controller configured to control the one or more valves such that the citric acid solution is first circulated through one of the first filter or the second filter for a first predetermined amount of time, and then the citric acid solution is circulated through the other of the first filter or the second filter for a second predetermined amount of time.

Aspect 10. The citric acid purification system according to aspect 9, further comprising an outlet line including an outlet valve, connected to the return line.

Aspect 11. The citric acid purification system according to any of aspects 9-10, wherein the first filter and the second filter each include a same membrane material.

Aspect 12. The citric acid purification system according to any of aspects 9-11, wherein the first filter is included in a first replaceable filter cartridge and the second filter is included in a second replaceable filter cartridge.

Aspect 13. The citric acid purification system according to any of aspects 9-12, wherein the first predetermined amount of time is approximately 4 hours and the second predetermined amount of time is approximately 2.5 hours.

Aspect 14. The citric acid purification system according to any of aspects 9-13, wherein the controller is further configured to determine when to replace one or more of the first filter and the second filter based on a loading of the one or more of the first filter and the second filter.

Aspect 15. The citric acid purification system according to aspect 14, wherein the loading of the one or more of the first filter and the second filter is determined based on a number of purification cycles performed since a most recent replacement of the one or more of the first filter and the second filter.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A method for purifying citric acid, comprising:
performing a first filtration of citric acid using a first filter; and
performing a second filtration, the second filtration being performed on the citric acid following the first filtration, using a second filter separate from the first filter.

2. The method of claim 1, wherein the first filter and the second filter each include a same filter membrane material.

3. The method of claim 2, further comprising selecting a filter having comparatively greater trapped metal ions to be used as the first filter, and a filter having comparatively fewer trapped metal ions to be used as the second filter.

4. The method of claim 3, further comprising replacing the first filter after completion of the first filtration.

5. The method of claim 1, wherein the performing of the first filtration includes recirculating the citric acid through a fluid circuit including the first filter for approximately 4 hours.

6. The method of claim 1, wherein the performing of the second filtration includes recirculating the citric acid through a fluid circuit including the second filter for approximately 2.5 hours.

7. The method of claim 1, wherein the citric acid is a 30% solution of citric acid.

8. The method of claim 1, wherein after the second filtration, the citric acid includes less than 5 parts per billion (ppb) of magnesium ions, and less than 10 ppb of calcium ions.

9. A citric acid purification system, comprising:
a tank configured to hold a citric acid solution;
a first filter configured to remove metal ions from the citric acid solution;
a second filter configured to remove metal ions from the citric acid solution, in parallel with the first filter;
one or more valves configured to direct a flow of a fluid from the tank to one of the first filter or the second filter;
a return line, configured to convey the fluid from the one of the first filter or the second filter to the tank; and
a controller configured to control the one or more valves such that the citric acid solution is first circulated through one of the first filter or the second filter for a first predetermined amount of time, and then the citric acid solution is circulated through the other of the first filter or the second filter for a second predetermined amount of time.

10. The citric acid purification system of claim 9, further comprising an outlet line including an outlet valve, connected to the return line.

11. The citric acid purification system of claim 9, wherein the first filter and the second filter each include a same membrane material.

12. The citric acid purification system of claim 9, wherein the first filter is included in a first replaceable filter cartridge and the second filter is included in a second replaceable filter cartridge.

13. The citric acid purification system of claim 9, wherein the first predetermined amount of time is approximately 4 hours and the second predetermined amount of time is approximately 2.5 hours.

14. The citric acid purification system of claim 9, wherein the controller is further configured to determine when to replace one or more of the first filter and the second filter based on a loading of the one or more of the first filter and the second filter.

15. The citric acid purification system of claim 14, wherein the loading of the one or more of the first filter and the second filter is determined based on a number of purification cycles performed since a most recent replacement of the one or more of the first filter and the second filter.
